(19) United States
(12) Patent Application Publication
(10) Pub. No.: US 2006/0280716 A1
(43) Pub. Date: Dec. 14, 2006

(54) Cationic aminosilicone emulsions

(76) Inventors: Anna Maria Czech, Cortlandt Manor, NY (US); Toshiko M. Maedo, Sao Paulo (BR)

Correspondence Address: GEAM - SILICONES - 60SI;IP LEGAL, ONE PLASTICS AVENUE, PITTSFIELD, MA, 01201, US

(21) Appl. No.: 11/150,043

(22) Filed: Jun. 10, 2005

(51) Int. Cl. A61K 8/89

(52) U.S. Cl. 424/70.122

(57) ABSTRACT

The present invention discloses a method for making cationic, aminofunctional silicone emulsions particularly cationic emulsions having a low volatile cyclic siloxane content. The method of the present invention comprises an acid catalyzed emulsion condensation reaction of a mixture comprising hydroxy terminated polydimethylsiloxane and an aminosilane under conditions that do not allow for the generation of equilibrium cyclic oligomers followed by conversion to a cationic emulsion.

### FIELD OF INVENTION

The field of the present invention relates to anionic emulsions of polymeric silicones or siloxanes, methods of making such emulsions and methods of converting anionic emulsions to cationic emulsions.

### BACKGROUND

Emulsion polymerization of silicone polymers to prepare high molecular weight emulsified silicone polymers is well known in the art. An early example of such technology has been disclosed in U.S. Pat. No. 2,891,920 and teaches the polymerization of octamethyltetrasiloxane (D₄) and other silicone oligomers using a strong base in the presence of quaternary ammonium compounds. This technology has been extended as taught in U.S. Pat. No. 3,294,725 to include the use of sulfonic acids to effect the polymerization of silicone oligomers. Polymerization of silicone oligomers in the presence of sulfonic acids is particularly effective in producing high molecular weight silicone polymers.

It is a well-recognized feature of the polymerization kinetics of silicone oligomers that low molecular weight polymers result from the initial ring opening polymerization. It has been found that control of the cooling step occurring after heating the reaction mixture containing the silicone oligomers to achieve the initial polymerization results in control of the degree of polymerization of the silicone. The extent of polymerization is kinetically controlled by approach to equilibrium and the temperature at which equilibrium is established. Generally, anionic polymerization catalysts allow for a rapid approach to equilibrium at all temperatures, in contrast to cationic polymerization catalysts where the approach to equilibrium is significantly slower. This results in a reliance on anionic polymerization catalysts in the commercial manufacture of silicone polymers, if high molecular weight polymers are desired.

The convenience afforded by the rapid reaction rates achievable with anionic polymerization catalysts, particularly in emulsion polymerization, limits the applications to which the resulting polymerized silicone emulsions may be used. While they may generally be employed in a variety of applications, including cosmetic applications, a change in the ionic balance of the emulsion frequently results in a break-up of the emulsion. Thus, treatment of an emulsion polymerized silicone emulsion, polymerized in the presence of an anionic polymerization catalyst, with a cationic surfactant for example will generally destroy the emulsion. As hair care products are typically required to possess cationic properties due to the weakly anionic nature of human hair, this leads to significant formulation difficulties in using emulsion polymerized high molecular weight silicone emulsions in human hair cosmetic and personal care products. In hair care applications, it is advantageous to use silicone emulsions containing cationic polymers for improved deposition on hair surfaces. It is also desirable to deposit high molecular weight silicone polymers on the hair because of the enhanced conditioning properties possessed by high molecular weight silicone polymers in contrast to low molecular weight silicone polymers.

The typical solution to this problem is to produce emulsion polymerized silicone emulsions that are polymerized in the presence of a cationic polymerization catalyst. However, emulsions produced in this fashion require long holding times to achieve the desired molecular weight of silicone polymer. Thus, ideally it would be desirable to be able to prepare high molecular weight silicone polymers by emulsion polymerization using anionic surfactants and then modify the product in some fashion to achieve the desired cationic properties that impart beneficial results to hair care formulations.

The simple expedient of adding cationic surfactants to high molecular weight silicone polymer emulsions that have been prepared using anionic surfactants is known to be a method of breaking up the emulsion. Adding a surfactant of opposite conjugate properties is a generally recognized technique for breaking emulsions. This is exemplified by a process for coagulating a grafted rubber compound as disclosed in U.S. Pat. No. 4,831,116 where the emulsion is prepared by a polymerization process in the presence of an anionic surfactant and the emulsion is broken and coagulated by the addition of a cationic surfactant.

There are some recent advances that indicate in certain isolated systems it is possible to preserve the emulsion when both cationic and anionic surfactants are present. As disclosed in U.S. Pat. No. 4,401,788, a vinylidene latex system produced by emulsion polymerization in the presence of an anionic surfactant tolerated the addition of a cationic surfactant. This particular system however, had a limited stability of the resulting emulsion. As disclosed in U.S. Pat. No. 5,045,576, anionic asphaltic emulsions can be converted to cationic emulsions through the addition of cationic surfactants in conjunction with a so-called steric stabilizer that prevents break-up or breakage of the emulsion. Neither of these systems deals with silicone polymer emulsions, nor would it be reasonable to expect that the techniques usable in lateces or asphalts would be directly transferable to silicone emulsions.

Aminofunctional silicone emulsions are widely utilized as hair conditioning ingredients in shampoos and conditioners, both rinse-off and leave-on, as well as textile softeners and treatments for woven and non-woven substrates. Hair care application prefer cationic amino emulsions as the cationic emulsifiers add to the deposition of the conditioning ingredient, the same applies to textile treatments that involve exhaustion. Numerous methods for making aminofunctional silicone emulsions are known in the art. These methods are generally classified in two categories: mechanical methods and emulsion polymerization/condensation methods. When employing mechanical emulsification, the polysiloxane does not undergo any reactions during the emulsification.

Emulsion polymerization methods comprise reacting a cyclooligosiloxane, such as octamethylcyclotetrasiloxane or decamethycyclopentasiloxane, in the presence of a catalyst, surfactant and water. During the reaction period there may be some form of agitation to provide adequate heat transfer for uniform temperature and to maintain uniform dispersion of the reactants. In emulsion polymerization, combination of oligocyclosiloxanes and reactive monomers or oligomers may be used to form copolymers in the resulting emulsions. Mechanical pre-emulsification of the silicone reactants may be used in emulsion polymerization methods. Similarly, emulsion condensation methods comprise reacting hydroxyterminated polydimethylsiloxane with above mentioned reactive monomers or oligomers to form copolymers. Methods for making polysiloxane emulsions by emulsion polymerization/condensation are provided, for example by U.S. Pat. No. 6,090,885, U.S. Pat. No. 4,784,665, U.S. Pat. No. 6,555,122 and U.S. Pat. No. 4,999,398.

### BRIEF SUMMARY

The method of the present invention comprises emulsion condensation of a mixture comprising hydroxyterminated polydimethylsiloxane and an aminosilane under anionic conditions followed by conversion to a cationic emulsion. The instant invention provides for a process for preparing a cationic silicone emulsion comprising the steps in order:

a) preparing a first emulsion consisting essentially of a
i) a hydroxy terminated polysiloxane;
ii) and aminosilane; and
iii) a non-ionic surfactant;

b) catalyzing the first emulsion by adding an acidic surfactant said acidic surfactant capable of acting as a catalyst and forming an anionic emulsion, the second emulsion, thereby, and

c) converting the second emulsion, the anionic emulsion, to a third emulsion, the cationic emulsion, by adding a cationic surfactant to the anionic emulsion in an amount sufficient to convert the anionic emulsion to the cationic emulsion.

The instant invention further provides for a method of making a cationic aminofunctional silicone emulsion having less than one weight percent volatile cyclic siloxane content. Additionally, the instant invention provides for personal care compositions utilizing the emulsions of the instant invention, particularly personal care compositions where it is desired for the composition to possess low levels of volatile cyclic siloxanes.

### DETAILED DESCRIPTION OF INVENTION

The present invention provides a process for making cationic aminofunctional silicone emulsions containing less than one weight percent of volatile cyclic siloxanes by emulsion condensation of a mixture comprising hydroxy terminated polysiloxane, an aminosilane and an acidic catalyst. Thus formed anionic emulsions of the aminofunctional polysiloxane are further converted to substantially stable cationic emulsions by addition of the sufficient amount of the cationic surfactant. Substantially stable aqueous emulsion is characterized by the dispersed particles that do not appreciably agglomerate during the typical shelf-life of the emulsion.

The method of the present invention comprises three steps in order:

1. Preparing an emulsified mixture, a premix, of hydroxyterminated polysiloxane and aminosilane using a nonionic surfactant, the surfactant preferably selected from, but not limited to ethoxylated aliphatic alcohols, fats, oils and waxes, carboxylic esters, such as esters of glycerin or polyethylene glycols and polyalkyleneoxide block copolymers. Nonionic surfactants commonly employed in such emulsions can include, for example, TERGITOL® surfactants available from Dow Chemical Co., using 10-30 weight % of the surfactant based on weight of the silicone phase. Hydroxyterminated polysiloxanes typically useful in the practice of instant invention have the general structure:
MₐD_{b}D′_{c}T_{d}Q_{f}
where

M=(HO)ⱼR¹ₖR²ₘSiO^{1/2}; where the k and m are zero positive and the sum of j+k+m is three and j is greater than or equal to one;

D=R³R⁴SiO_{2/2};

D′=R⁵R⁶SiO_{2/2};

T=R⁷SiO_{3/2};

Q=SiO_{4/2};

where each R¹, R², R³, R⁴, R⁵, R⁶ and R⁷ are each independently chosen from the group of C1 to C40 monovalent hydrocarbon radicals and the subscripts a, b, c, d, e and f are chosen so that the viscosity of the polysiloxane ranges from about 10 to about 1000 cSt, particularly from about 30 to about 500 cSt, more particularly from about 50 to about 250 cSt, and most particularly from about 80 to about 150 cSt, and all sub-ranges therebetween.

Hydroxyterminated polydimethylsiloxanes (R^{x}=CH₃) are commercially available under trade names L-9000 from GE Advance Materials or Q1-2343 from Dow Coming Corp.

Aminosilanes useful in the instant invention have the general structure:
(OR⁸)₃₋ₓ Si(R⁹)ₓ—R¹⁰—NH—Y, where Y is selected from the group consisting of hydrogen, alkyl, cycloalkyl, aryl or arylalkyl group containing 1-18 carbon atoms, and —R¹¹—(R¹²)ₓ Si(OR¹³)₃₋ₓ. R⁸, R⁹, R¹² and R¹³ are the same or different and selected from the group consisting of C1 to C6 monovalent alkyl or C6 to C10 monovalent aryl, x=0-2. R¹⁰ and R¹¹ are divalent radicals selected from the group consisting of C3 to C12 linear or branched chains, phenylene groups or a combination of hydrocarbon and aromatic units. These aminosilanes are exemplified by, but not limited to aminopropyltriemethoxysilane, 4-aminobutyltriethoxysilane, aminoethylaminoisobutylmethyldiethoxysilane, p-aminophenyltrimethoxysilane, N-cyclohexylaminopropyltrimethoxysilane, bis(trimethoxysilylpropyl)amine and bis((3-trimethoxxysilyl)propyl)-ethylenediamine.

Typically the aminosilane is present in an amount of 0.1 to 10 wt % based on the total weight of the oil phase of the emulsion, preferably 0.5-1.5% based on the weight of the premix. The premix may contain one or more aminosilanes.

2. Catalyzing the nonionic emulsion with acid catalyst surfactant such as for example, surface active sulfonic acids, which can be substituted with alkyl, aralkyl, or aryl radicals. A particularly preferred acid catalyst surfactant is dodecylbenzenesulfonic acid. An effective amount of the acid catalyst is in the range between 0.25% by weight and about 5% by weight based on the weight of the pre-mix. Allowing emulsion condensation to take place at room temperature over 24-48 hours. Neutralizing the emulsion with an inorganic base or a tertiary amine, such as, but not limited to sodium, potassium or ammonium hydroxide, triethanolamine and 2-amino-2-methylpropanol.

3. Converting the anionic emulsion into a cationic emulsion by a slow addition of a sufficient amount of a cationic surfactant. Typically the recommended molar ratio of the cationic to anionic surfactant is from about 1.1:1 to about 5:1, specifically from about 1.25:1 to about 2.5:1, and most specifically from about 1.4:1 to about 1.6:1. Cationic surfactants that may be employed in the present invention can be selected from, but not limited to alkyltrimethylammonium, dialkyldimethylammonium, alkylpyridinium, benzalkonium or imidazolinium halides, preferably chloride or bromides.

Emulsions made by the process of the present invention contain less than five weight percent volatile cyclic siloxane; particularly less than three weight percent of volatile cyclic siloxane, more particularly less than two weight percent volatile cyclic siloxane and most particularly less than weight percent volatile cyclic siloxane. As used herein the term volatile cyclic siloxane means a siloxane having the formula: (R¹⁴R¹⁵SiO)ₘ where each R¹⁴ and R¹⁵ are independently selected from the group of C1 to C10 monovalent hydrocarbon radicals, m ranges from 3 to 8. The word volatile as used herein means having a measurable vapor pressure below 760 torr at 25° C. and 760 torr pressure.

Commercially available examples of such cationic surfactants are: Barquat MB-80, Barquat MX-50, Empigen BAC 80/S, Varisoft 300 and Varisoft TA100.

The emulsions produced by the method of this invention typically contain 10 to 70 weight % of an aminofunctional polysiloxane polymer, preferably 20 to 50 weight %.

Emulsions formed by the method of the present invention can be applied onto the substrates such as by spraying, dipping or kiss roll application or other application method typically employed in fiber, hair or textile treatment. The substrate which can be treated with the copolymers of the present invention is exemplified by natural fibers such as hair, cotton, silk, flax, cellulose, paper (including tissue paper) and wool; synthetic fibers such as polyester, polyamide, polyacrylonitrile, polyethylene, polypropylene and polyurethane; and inorganic fibers such as glass or carbon fibers.

In general the emulsions are applied onto hair, fiber or textile such that up to 5%, preferably 0.01 to 2.5% of the aminofunctional silicone by weight of the dry substrate remains on the substrate. Optionally other additives, commonly used to treat hair or textile substrates can be employed along with the copolymers of the present invention, including but not limited to additional surfactants, deposition polymers, quaternary conditioning agents, curing resins, preservatives, dyes, colorants, formularies.

Furthermore, emulsions made by the method of the present invention may be used in personal care formulations, including cleansers, body washes, soaps, lotions, creams, shaving cream, hair sprays, conditioners, shampoos, deodorants, moisturizers, and sunblocks. They can be formulated into these or other products together with one or more anionic surfactants, one or more amphoteric surfactants, one or more nonionic surfactants, one or more cationic surfactants, and/or one or more deposition polymers or thickeners.

Moreover, emulsions made by the method according to the present invention may be used in personal care formulations, including rinse-off and leave-on hair conditioners, conditioning shampoos, cleansers, body washes, soaps. They can be formulated into these or other products together with one or more cationic surfactants, one or more anionic surfactants, one or more amphoteric surfactants, one or more nonionic surfactants, and/or one or more deposition polymers or thickeners.

### EXPERIMENTAL

Acid Catalyzed Emulsion Condensation

150 g of Trideceth-12 and 180 g of water were placed in a vessel and mixed for 15 minutes, using Cowels disperser at 1300-1500 rpm until homogeneous. Hydroxyterminated polydimethylsiloxane (1050 g, D4 content 0.25%) and bis(trimethoxysilylpropyl)amine (7.35 g) were pre-blended in a separate vessel. The pre-blend was added in portions to the surfactant and water soap and mixed for 15 minutes at 1300-1500 rpm after each addition. Once pre-blend was added, mixing continued until white, flaky grease was formed. Balance water (1454 g) was then added slowly to the grease followed by 30 g of dodecylbenzenesulfonic acid. Vessel contents were mixed for additional 30 minutes and kept at room temperature for 48 hours. The emulsion was then neutralized with triethanolamine to pH 8, followed by addition of Cetrimonium Chloride (50% in ethanol). Final emulsion had solids content of 44.26% and D4 content was 0.16% and D5 content was 0.04%.

Acid Catalyzed Emulsion Condensation

In this example, 50 g of nonylphenol ethoxylate-15 EO were mixed with 40 g of water to form soap. To this, 350 g of hydroxyterminated polydimethysiloxane with 93.50% solids and 1.11% of D4 content pre-blended with 2.45 g of bis(trimethoxysilylpropyl)amine were added in portions and mixed until obtain a grease phase followed by slow addition of 540 g of water. After obtaining the emulsion, 7.5 g of dodecylbenzenesulfonic acid were added and mixed. After allowing sufficient time for polymerization, the emulsion was neutralized with trietanolamine q,s,p to pH 8.0. The solids of the emulsion were of 38.0% and D4 content was of 0.21% and D5 content was 0.09%.

### COMPARATIVE EXAMPLE A

### Base Catalyzed Emulsion Condensation

40 g of Trideceth-12, 30 g of Cetrimonium Chloride 50% in alcohol and 43 g of water were mixed at moderate speed using Cowles disperser to form soap. To this blend, 350 g of hydroxy terminated polydimethylsiloxane (1.11% D4) pre-blended with 2.45 g of bis(trimethoxysilylpropyl)amine was added in small portions until grease was formed followed by slow addition of 423.5 g of water and 2.0 g of the sodium hydroxide pre-dissolved in 100 g of water to reach pH of 12.1. The emulsion was kept for 5 days at ambient temperature to react, then neutralized acetic acid to pH 7.0. The solids content was adjusted with water to 38%; D4 content was 1.2%, D5 content was 0.39%, a result above 1.0 wt. % volatile siloxane.

### COMPARATIVE EXAMPLE B

### Base/Acid Catalyzed Emulsion Condensation

50 g of Trideceth-12, 30 g of Cetrimonium Chloride 50% in alcohol and 60 g of water were mixed at moderate speed in Cowles disperser until obtaining a soap. Then, 350 g of hydroxy terminated polydimethysiloxane pre-mixed with 2.45 g of amino silane were added in portions and mixed until uniform. After homogenization, 400 g of water were added slowly followed by 2.0 g of NaOH dissolved in 100g of water. After 5 days, emulsion was acidified with hydrochloric acid to pH 1.85 and kept at 50° C. for additional 5 days. After neutralization, D4 content was 1.23% and D5 was 0.37%, a result above 1.0 wt. % volatile siloxane.

### APPLICATION DATA

Conditioning Shampoo and Rinse-Off Conditioner

| Shampoo Base | | | Conditioner Base | | |
|---|---|---|---|---|---|
| Phase | Components | % | Phase | Components | % |
| A | Sodium Laureth-2 | 30 | A | Cetearyl Alcohol | 4.0 |
| | Sulfate | | | Ceteareth-20 | 2.0 |
| | PEG-150 | 1.0 | | Methylparaben | 0.1 |
| | Distearate | | B | Water | QS to |
| B | Cocamidopropyl | 3.0 | | | 100 |
| | Betaine | | C | Cetrimonium | 2.5 |
| | Cocoamide DEA | 3.0 | | Chloride (25%) | |
| | Water | QS to | | | |
| | | 100 | | | |
| C | Citric Acid | pH | | | |
| | | 5.5-6.0 | | | |

Mixing Instructions for the Shampoo Base: Sodium Laureth Sulphate and PEG-150 Distearate were heated in a water bath and mixed until uniform. Remaining ingredients: Cocoamidopropyl Betaine, Cocoamide DEA and water were added with mixing one ingredient at a time. The pH was then adjusted to 5.5-6.0 with citric acid.

Conditioning shampoos were prepared by post adding sufficient amounts of the emulsion from example 1.6.1.1 to amount to 0.5 and 1% silicone actives. Thus prepared conditioning shampoos were stable at 50° C. and ambient for a period of 20 days.

Mixing Instructions for the Rinse-off Conditioner Base: An oil phase consisting of Cetearyl Alcohol and Ceteareth-20 was heated to 70° C. in water bath. Once homogeneous, it was added to water pre-heated to 70° C. and mixed at moderate speed until cooled to 40° C. and Cetrimonium Chloride 25% in alcohol was added.

Silicone containing rinse-off conditioners were prepared by post adding sufficient amounts of the emulsion from example 1.6.1.1 to amount to 0.5 and 1% silicone actives followed by homogenization. Stability of the conditioner was tested at 50° C. and at ambient for 20 days.

Hair Treatment and Testing

Damaged (colored) hair tresses 25 cm long weighting 5 g each were tested. Following tests were performed: Wet and dry combability (manually), gloss and hand panel to evaluate conditioning benefits of the emulsions of the present invention.

Shampoo and Rinse-Off Conditioner Test Procedures

1. Wet three tresses with running tap water at 40-42° C.

2. Apply 1 ml (by syringe) of the test solution to each hair tress individually and work in for 30-45 sec.

3. Rinse each tress individually with running tap water at 40-42° C. for 30-35 sec and then blot dry.

4. Hang each tress on a calibrated chart and comb hair from top to bottom (using the large tooth end) until comb snags. Wet combability is measured as a number of cm comb travels. The tress is also inspected for wet feel and wet appearance and recorded in notebook.

5. The snags are combed out of the tress and it is placed under hair dryer for 1-1.5 hours.

6. The tress is again placed on the chart and the comb run through from top to bottom until it snags, thus measuring dry combability.

7. The tress is then combed quickly ten times to produce a static charge in order to measure flyaway. The flyaway is reported as the width in cm of the mass of hair bundle taken away from the total with of the entire tress.

8. The dry feel is the tested by hand panel.

Wet Combability (cm)—Detangling

Higher numbers mean better detangling

TABLE 1

| Hair Tresses | Shampoo | Conditioner |
|---|---|---|
| Base | 3.0 | 7.0 |
| Silicone 0.5% | 3.5 | 12.0 |
| Silicone 1.0% | 6.0 | 15.0 |

Hair tresses treated with silicone containing formulations were easier to comb than tresses treated with Base formulations
Dry Combability (cm)—Detangling

Higher numbers mean better detangling

TABLE 2

| Hair Tresses | Shampoo | Conditioner |
|---|---|---|
| Base | 6.0 | 11.0 |
| Silicone 0.5% | 7.0 | 12.0 |
| Silicone 1.0% | 9.0 | 14.0 |

Hair tresses treated with silicone containing formulations were easier to comb than tresses treated with Base formulations

Gloss

TABLE 3
Higher numbers mean higher gloss
Average 6 readings

| Hair Tresses | Shampoo | Conditioner |
|---|---|---|
| Base | 0.85 | 0.77 |
| Silicone 0.5% | 1.02 | 1.30 |
| Silicone 1.0% | 1.32 | 1.42 |

Hair tresses treated with silicone containing formulations were more glossy than tresses treated with Base formulations

Fly Away (cm)

TABLE 4
Lower numbers mean less static build-up

| Hair Tresses | Shampoo | Conditioner |
|---|---|---|
| Base | 5.5 | 4.5 |
| Silicone 0.5% | 5.0 | 3.0 |
| Silicone 1.0% | 4.5 | 2.5 |

Hair tresses treated with silicone containing formulations had less static build-up than tresses treated with Base formulations.

The foregoing examples are merely illustrative of the invention, serving to illustrate only some of the features of the present invention. The appended claims are intended to claim the invention as broadly as it has been conceived and the examples herein presented are illustrative of selected embodiments from a manifold of all possible embodiments. Accordingly it is Applicants' intention that the appended claims are not to be limited by the choice of examples utilized to illustrate features of the present invention. As used in the claims, the word “comprises” and its grammatical variants logically also subtend and include phrases of varying and differing extent such as for example, but not limited thereto, “consisting essentially of” and “consisting of.” Where necessary, ranges have been supplied, those ranges are inclusive of all sub-ranges there between. It is to be expected that variations in these ranges will suggest themselves to a practitioner having ordinary skill in the art and where not already dedicated to the public, those variations should where possible be construed to be covered by the appended claims. It is also anticipated that advances in science and technology will make equivalents and substitutions possible that are not now contemplated by reason of the imprecision of language and these variations should also be construed where possible to be covered by the appended claims. All United States patents referenced herein are herewith and hereby specifically incorporated by reference.

Having described the invention that which is claimed is:

1. A process for preparing a cationic silicone emulsion comprising the steps in order:
a) preparing a first emulsion consisting essentially of a
i) a hydroxy terminated polysiloxane;
ii) and aminosilane; and
iii) a non-ionic surfactant;
b) catalyzing the first emulsion by adding an acidic surfactant said acidic surfactant capable of acting as a catalyst and forming an anionic emulsion, the second emulsion, thereby; and
c) converting the second emulsion, the anionic emulsion, to a third emulsion, the cationic emulsion, by adding a cationic surfactant to the anionic emulsion in an amount sufficient to convert the anionic emulsion to the cationic emulsion.

2. The third emulsion of claim 1 comprising less than about 1.0 weight percent cyclic volatile siloxanes.

3. The first emulsion of claim 1 wherein said hydroxy terminated polysiloxane has the formula:
MₐD_{b}D′_{c}T_{d}Q_{f}
where
M=(HO)ⱼR¹ₖR²ₘSiO_{1/2};, where the k and m are zero positive and the sum of j+k+m is three and j is greater than or equal to one;
D=R³R⁴SiO_{2/2};
D′=R⁵R⁶SiO_{2/2};
T=R⁷SiO_{3/2};
Q=SiO_{4/2};
where each R¹, R², R³, R⁴, R⁵, R⁶ and R⁷ are each independently chosen from the group of C1 to C40 monovalent hydrocarbon radicals and the subscripts a, b, c, d, e and f are chosen so that the viscosity of the polysiloxane ranges from about 10 to about 1000 cSt.

4. The first emulsion of claim 1 wherein said aminosilane has the formula:
(OR⁸)₃₋ₓSi(R⁹)ₓ—R¹⁰—NH—Y,
where Y is selected from the group consisting of hydrogen, alkyl, cycloalkyl, aryl or arylalkyl group containing 1-18 carbon atoms, and —R¹¹—(R¹²)ₓSi(OR¹³ )₃₋ₓ. R⁸, R⁹, R12 and R¹³ are the same or different and selected from the group consisting of C1 to C6 monovalent alkyl or C6 to C 10 monovalent aryl, x=0-2. R₁₀ and R¹¹ are divalent radicals selected from the group consisting of C3 to C12 linear or branched chains, phenylene groups or a combination of hydrocarbon and aromatic units.

5. The first emulsion of claim 1 wherein said non-ionic surfactant is selected from the group consisting of ethoxylated aliphatic alcohols, fats, oils and waxes, carboxylic esters, and polyalkyleneoxide block copolymers.

6. The first emulsion of claim 3 wherein said aminosilane has the formula:
(OR⁸)₃₋ₓSi(R⁹)ₓ—R¹⁰—NH—Y,
where Y is selected from the group consisting of hydrogen, alkyl, cycloalkyl, aryl or arylalkyl group containing 1-18 carbon atoms, and —R¹¹—(R¹²)ₓSi(OR¹³)₃₋ₓ. R⁸, R⁹, R¹² and R¹³ are the same or different and selected from the group consisting of C 1 to C6 monovalent alkyl or C6 to C10 monovalent aryl, x=0-2. R¹⁰ and R¹¹ are divalent radicals selected from the group consisting of C3 to C12 linear or branched chains, phenylene groups or a combination of hydrocarbon and aromatic units.

7. The first emulsion of claim 6 wherein said non-ionic surfactant is selected from the group consisting of ethoxylated aliphatic alcohols, fats, oils and waxes, carboxylic esters, and polyalkyleneoxide block copolymers.

8. The first emulsion of claim 6 wherein said aminosilane is selected from the group consisting of aminopropyltriemethoxysilane, 4-aminobutyltriethoxysilane, aminoethylaminoisobutylmethyldiethoxysilane, p-aminophenyltrimethoxysilane, N-cyclohexylaminopropyltrimethoxysilane, bis(trimethoxysilylpropyl)amine and bis ((3-trimethoxxysilyl)propyl)-ethylenediamine.

9. The first emulsion of claim 7 wherein each R¹, R², R³, R⁴, R⁵, R⁶ and R⁷ is methyl.

10. The third emulsion of claim 9 comprising less than about 1.0 weight percent cyclic volatile siloxanes.

11. A personal care composition comprising an aminosilane emulsion, said aminosilane emulsion comprising:
a) an acidic surfactant;
b) an amino silane; and
c) a cationic surfactant
wherein said personal care composition comprises less than about 1.0 weight percent cyclic volatile siloxanes.

12. The personal care composition of claim wherein said personal care composition is selected from the group consisting of rinse-off hair conditioners, leave-on hair conditioners, conditioning shampoos, cleansers, body washes, and soaps.
